(19) United States
(12) Patent Application Publication
(10) Pub. No.: US 2023/0067368 A1
(43) Pub. Date: Mar. 2, 2023

(54) Catheter Adapter with Flexible Strain Relief

(71) Applicant: Becton, Dickinson and Company, Franklin Lakes, NJ (US)

(72) Inventors: John M. Lackey, West Valley City, UT (US); Shaun Staley, Murray, UT (US); Ralph L. Sonderegger, Farmington, UT (US); Dorien Gemma Butter, Millcreek, UT (US); Alison Nicole Thompson, Salt Lake City, UT (US); Nathan Aaron Mitchell, Murray, UT (US)

(21) Appl. No.: 17/897,358

(22) Filed: Aug. 29, 2022

Related U.S. Application Data
(60) Provisional application No. 63/239,164, filed on Aug. 31, 2021.

(51) Int. Cl. A61M 39/10

(52) U.S. Cl. CPC A61M 39/10; A61M 2039/1077; A61M 2039/1033

(57) ABSTRACT

A catheter adapter including a housing and an extension portion extending from the distal end of the housing and defining a passageway configured to receive a catheter. The first material of the housing is more flexible than the second material of the extension portion. The catheter adapter may further include a body contact portion comprising an upper surface corresponding to a surface of the housing and a lower surface defining an outer surface of the catheter adapter. The housing may include a body, an access port extending from the body, and a plurality of protrusions extending from the body and/or the access port. The body contact portion covers at least a portion of the housing, and the protrusions are embedded in and are completely surrounded by the body contact portion. The catheter adapter may further include a connection portion connecting the extension portion to the body contact portion.

### CROSS-REFERENCE TO RELATED APPLICATIONS

This application claims priority to U.S. Provisional Patent Application No. 63/239,164 filed Aug. 31, 2021, the disclosure of which is hereby incorporated by reference in its entirety.

### BACKGROUND OF THE INVENTION

### Field of the Invention

The present invention is directed to a catheter adapter, more specifically, to a catheter adapter with a flexible member that reduces strain on the catheter when the catheter has been inserted into a blood vessel of a patient and a vascular access assembly including the catheter adapter.

### Description of Related Art

Catheters are frequently utilized to administer fluids into and out of the body and to draw blood samples from the body. Patients in a variety of settings, including in hospitals and in home care, receive fluids, pharmaceuticals, and blood products via a vascular access device inserted into a patient's vascular system. Catheters of various types and sizes have been used extensively in a variety of procedures including, but not limited to, treating an infection, providing anesthesia or analgesia, providing nutritional support, treating cancerous growths, maintaining blood pressure and heart rhythm, and many other clinical uses. A common vascular access device is a plastic catheter that is inserted into a patient's blood vessel. The catheter length may vary from a few centimeters for peripheral access to many centimeters for central access. The catheter is commonly incorporated into a catheter adapter to aid in the ease of use, accessibility, and utility of the catheter. A catheter adapter houses one end of the catheter such that one end of the catheter is supported by the catheter adapter and the body and tip of the catheter extends beyond a first end of the catheter adapter. A catheter adapter generally further includes an access port adapted to receive additional infusion components for use with the catheter. For example, the access port of the catheter adapter may include a set of threads for attaching an intravenous line or for coupling a syringe to the catheter adapter thereby providing access to the patient's vasculature via the attached catheter.

The catheter may be inserted transcutaneously. When inserted transcutaneously, the insertion of the catheter is commonly aided by an introducer needle. The introducer needle is commonly housed inside the lumen of the catheter such that the gauge of the needle approximates the inner diameter of the catheter. The needle is positioned within the catheter such that the needle tip extends beyond the tip of the catheter whereby the needle is used to penetrate the patient's blood vessel and provide an opening for insertion of the catheter.

During insertion into the body of a patient, the needle and catheter generally approach the patient's blood vessel at an angle of about 30° where the needle initially punctures the patient's epidermis and then continues into the blood vessel. Once the needle and catheter tip enter the patient's blood vessel, the needle and catheter are then repositioned so that the needle and catheter are brought into a position generally parallel with the patient's blood vessel so that the needle and catheter may be inserted into the lumen of the patient's blood vessel. When the catheter has been properly positioned within the patient's blood vessel, the needle is removed from the lumen of the catheter and the catheter adapter is secured to the patient to prevent premature removal of the catheter.

The catheter adapter may be secured to the patient by fastening the catheter adapter to the patient's skin via tape, a securement device, and/or a securement dressing. When securing the catheter adapter to the patient's skin, the root region of the catheter immediately exiting the catheter adapter must arch to accommodate the transition of the catheter from the generally parallel, secured orientation of the catheter adapter, to the insertion angle of the catheter.

Referring to FIG. 1, an extended section of catheter 12 may be left between the patient and the catheter adapter 10 to allow for transitional arching of the catheter 12. This exposed, archable length of catheter 12 biases the catheter 12 towards the patient's skin. The portion of the catheter 12 extending from the catheter adapter 10 may generally follow an S-shaped profile. The catheter 12, however, may kink slightly at the bends of the S-shaped profile, which can occlude the catheter 12 and/or inhibit the passage of instruments through the catheter 12. For example, the S-shaped profile and/or kinks in the catheter can pose a challenge for line draw devices which utilize the catheter 12 and pass through the catheter 12 to open up a fluid path for blood draw.

### SUMMARY OF THE INVENTION

The present invention is directed to a catheter adapter comprising a housing having a proximal end and a distal end and an extension portion extending from the distal end of the housing. The extension portion has a proximal end and a distal end and defines a passageway configured to receive a catheter. The housing comprises a first material and the extension portion comprises a second material, and the second material is more flexible than the first material.

The first material maybe a polymer and the second material may be an elastomer. The second material may have a hardness of 50-70 shore A.

At least a portion of the housing may be cylindrical, the extension portion may be cylindrical. An outer diameter of the extension portion may be the same as an outer diameter of the distal end of the housing or may be less than an outer diameter of the distal end of the housing.

At least a portion of the extension portion may be transparent. A plurality of radial channels and/or a plurality of axial channels may be provided in an outer surface of the extension portion. The extension portion may have a top portion and a bottom portion positioned opposite the top portion, where the bottom portion is configured to face a skin surface of a patient, and a plurality of radial channels provided only on the bottom portion of the extension portion. Alternatively, a helical channel may be provided in the outer surface of the extension portion.

The catheter adapter may further comprise a body contact portion having an upper surface corresponding to a surface of the housing and a lower surface defining an outer surface of the catheter adapter.

The housing may comprise a body having a proximal end and a distal end, an access port extending from the body, and a plurality of protrusions extending from the body and/or the access port. The body contact portion covers at least a portion of the housing, and the protrusions are embedded in and are completely surrounded by the body contact portion.

The catheter adaptor may further comprise a connection portion connecting the extension portion to the body contact portion. The housing may be made from a first material, and the extension portion, the body contact portion, and the connection portion may be made from a second material, where the second material is more flexible than the first material.

The catheter adaptor may further comprise an extension tip extending from the distal end of the body of the housing, where the extension portion surrounds at least a portion of the extension tip. The extension tip may comprise a central portion defining a lumen and a flange that surrounds the central portion with the central portion extending distally beyond the flange. A notch may be provided in the extension tip. The notch may be substantially wedge-shaped. If a connection portion connecting the extension portion to the body contact portion is provided, the connection portion may extend through and substantially fill the notch in the extension tip of the housing.

The catheter adaptor may further comprise a recess in an outer surface of the body of the housing in an area covered by the body contact portion. When an extension tip extending from the distal end of the body of the housing is provided and a notch is provided in the extension tip, the recess may be connected to the notch in the extension tip, such that a sidewall of the notch is continuous with a sidewall of the recess. The recess may extend around an outer circumference of the body of the housing for an angular distance equal to an angular distance defined by the notch in the extension tip, and a circumferential width of a distal end of the recess may be less than a circumferential width of the proximal end of the recess. The recess may extend along a length of the body of the housing from the notch in the extension portion to a location proximal to the access port. At least one protrusion extending from the outer surface of the body of the housing may be provided in the recess.

The catheter adaptor may further comprise a flange extending laterally from an outer surface of the body of the housing, where the body contact portion abuts the flange. When a recess is provided in an outer surface of the body of the housing in an area covered by the body contact portion, the protrusions extending from the body of the housing may be positioned between the flange of the housing and the recess and circumferentially spaced from the recess.

At least one of the plurality of protrusions may extend from the body of the housing and at least one of the plurality of protrusions may extend from the access port. The protrusions may have a flat, wing-shaped configuration. When a flange extending laterally from an outer surface of the body of the housing is provided, a lateral distance that the protrusions extend from the housing may be greater than a lateral distance that the flange extends from the housing. A lateral distance that the protrusions of the plurality of protrusions positioned near the distal end of the body of the housing extend from the housing may be less than a lateral distance that the protrusions of the plurality of protrusions positioned nearer the proximal end of the body of the housing and/or a lateral distance that the protrusions of the plurality of protrusions positioned on the access port of housing extend from the housing. A width, in a longitudinal direction of the housing, of the protrusions of the plurality of protrusions positioned near the distal end of the body of the housing may be less than a width, in the longitudinal direction of the housing, of the protrusions of the plurality of protrusions positioned nearer the proximal end of the body of the housing and/or a width, in the longitudinal direction of the housing, of the protrusions of the plurality of protrusions positioned on the access port.

A first wing member may extend from a first side of the housing and a second wing member may extend from a second side of the housing. The first and second wing members may each be formed from the second material and may extend from opposite sides of the body contact portion.

The catheter adaptor may further comprise a catheter extending from the extension portion.

The present invention is also directed to a vascular access assembly comprising the above-described catheter adapter, a catheter, and a needle assembly. The vascular access assembly may further comprise an extension tube connected to an access port extending from the housing of the catheter adaptor and a fluid connector associated with the extension tube. The fluid connector may comprise at least one needle-free connector for connecting a fluid source or a blood collection device to the extension tube.

### BRIEF DESCRIPTION OF THE DRAWINGS

FIG. 1 is a partial cross-sectional view of a catheter adapter and catheter, showing the catheter inserted into a blood vessel of a patient;

FIG. 2 is a perspective view of a vascular access device according to one aspect or embodiment of the invention;

FIG. 3 is a side view of a catheter adapter according to one aspect or embodiment of the invention;

FIG. 4 is a side view of a catheter adapter according to one aspect or embodiment of the invention;

FIG. 5 is a side view of a catheter adapter according to one aspect or embodiment of the invention;

FIG. 6 is a side view of a catheter adapter according to one aspect or embodiment of the invention;

FIG. 7 is a side view of a catheter adapter according to one aspect or embodiment of the invention;

FIG. 8 is a side view of a catheter adapter according to one aspect or embodiment of the invention;

FIG. 9 is a side view of a catheter adapter according to one aspect or embodiment of the invention;

FIG. 10 is a perspective view of a catheter adapter according to one aspect or embodiment of the invention;

FIG. 11 is a partial cross-sectional view of a catheter adapter and catheter according to one aspect or embodiment of the invention;

FIG. 12 is a partial cross-sectional view of a catheter adapter and catheter according to one aspect or embodiment of the invention;

FIG. 13 is a top view of a housing of a catheter adapter according to one aspect or embodiment of the invention;

FIG. 14 is a top view of a housing of a catheter adapter according to one aspect or embodiment of the invention;

FIG. 15 is a bottom view of a housing of a catheter adapter according to one aspect or embodiment of the invention;

FIG. 16 is a bottom view of a housing of the catheter adapter of FIG. 15;

FIG. 17 is a bottom view of a housing of the catheter adapter of FIG. 15;

FIG. 18 is a perspective side end view of a housing of a catheter adapter according to one aspect or embodiment of the invention;

FIG. 19 is a perspective bottom end view of a housing of a catheter adapter according to one aspect or embodiment of the invention;

FIG. 20 is a cross-sectional view of a catheter adapter according to one aspect or embodiment of the invention; and

FIG. 21 is a top view of a catheter adapter according to one aspect or embodiment of the invention.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DESCRIPTION OF THE INVENTION

For purposes of the description hereinafter, the terms “upper”, “lower”, “right”, “left”, “vertical”, “horizontal”, “top”, “bottom”, “lateral”, “longitudinal”, “proximal”, “distal” and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

“Including”, “such as”, “for example” and like terms means “including/such as/for example but not limited to”. The terms “first”, “second”, and the like are not intended to refer to any particular order or chronology, but refer to different conditions, properties, or elements.

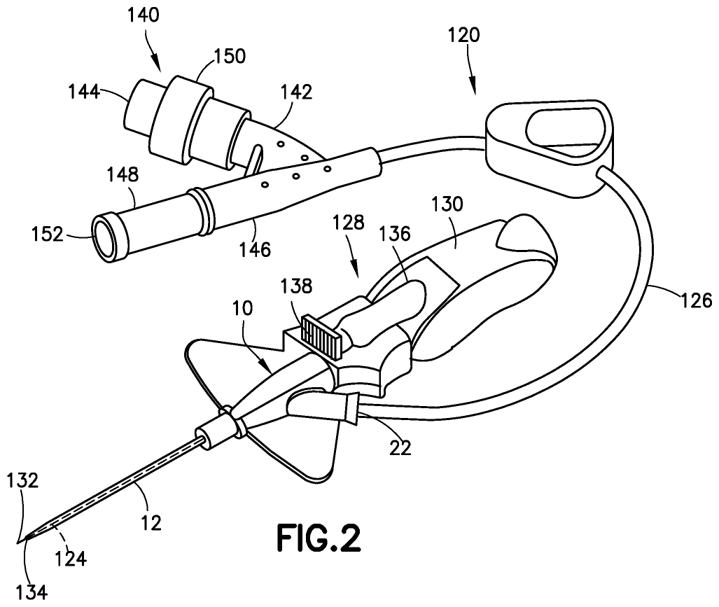

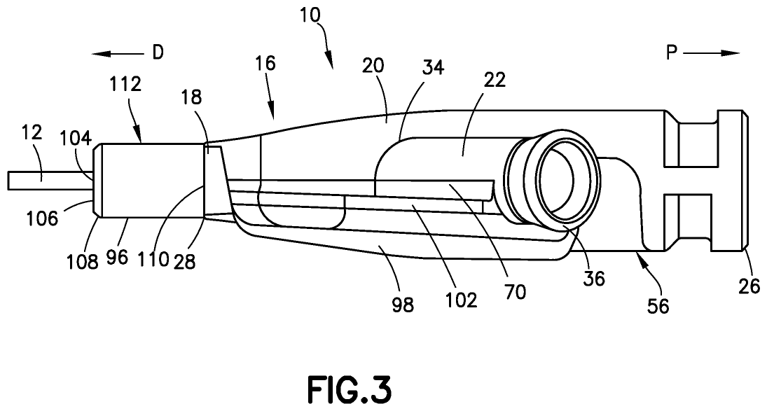

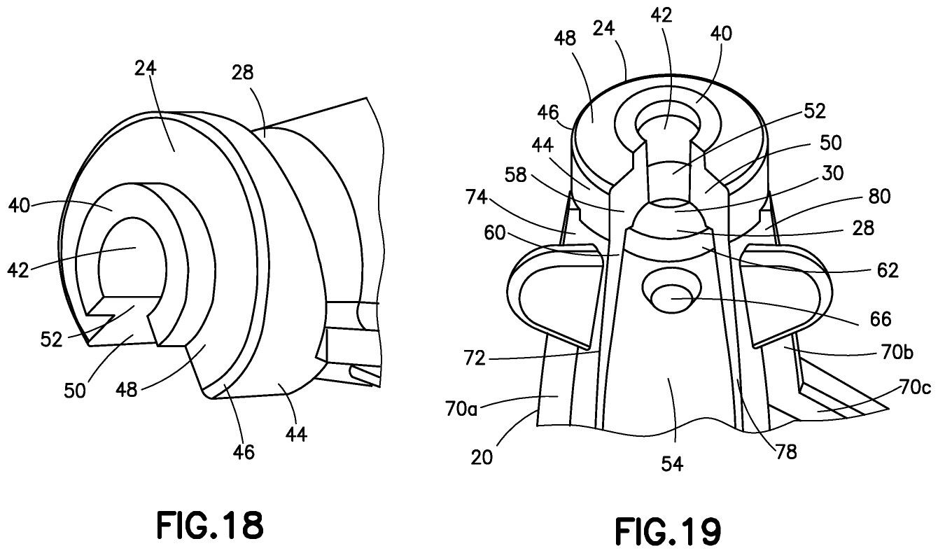

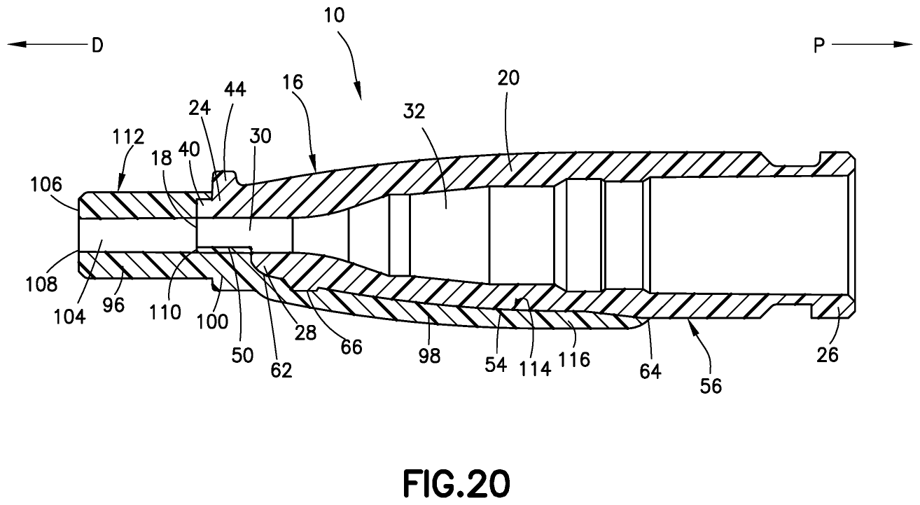

The present invention is directed to a catheter adapter 10 (FIGS. 3-21) and a vascular access assembly 120 (FIG. 2) including the inventive catheter adapter 10 and a needle assembly 128 and/or a fluid connector 140.

The catheter adapter 10 aids insertion of a flexible catheter 12 into a blood vessel 14 of a patient and acts to secure the catheter 12 and to provide connection between the catheter 12 and an intravenous fluid source to allow introduction of the intravenous fluid into the blood vessel 14 of the patient or connection to a blood collection device to withdraw a blood sample from the blood vessel 14. The vascular access assembly 120 provides access to the patient's blood vessel 14 via an introducing needle 124 over which the catheter 12 of the catheter adapter 10 is threaded. Upon successful insertion of the needle 124/catheter 12 into the blood vessel 14, the needle 124 is removed and the catheter 12 remains in the blood vessel 14.

As referred to herein, “proximal” refers to the end of the catheter adaptor 10 and the direction indicated by the arrow “P” in the figures and “distal” refers to the end of the catheter adaptor 10 and the direction indicated by the arrow “D” in the figures.

As shown in FIGS. 3-21, the catheter adapter 10 comprises housing 16 and a flexible nose extension portion 96 and a body contact portion 98 securely attached to the housing 16.

The housing 16 comprises a body 20, a lateral access port 22, and an optional extension tip 24.

The body 20 of the housing 16 has a proximal end 26 and a distal end 28. The proximal end 26 of the body 20 is adapted for attachment to the needle assembly 128, and a catheter 12 extends from an opening 30 in the distal end 28 of the body 20. The body 20 defines a passageway 32 in fluid communication with the catheter 12.

A lateral access port 22 extends from the body 20 of the housing 16. The access port 22 has a central longitudinal axis that is at an angle to the central longitudinal axis of the body 20 of the housing 16. The access port 22 has a first end 34 that is integral with the body 20 of the housing 16 and a second end 36 adapted for connection to an intravenous fluid source or a blood collection device. The access port 22 defines a passageway. The passageway is in fluid communication with the passageway 32 of the body 20 of the housing 16 at the first end 34 of the access port 22 and in fluid communication with the attached intravenous fluid source or blood collection device at the second end 36 of the access port 22. The second end 36 of the access port 22 may be configured for connection to an extension tube 126 for establishing fluid communication between an intravenous fluid source and the catheter adapter 10.

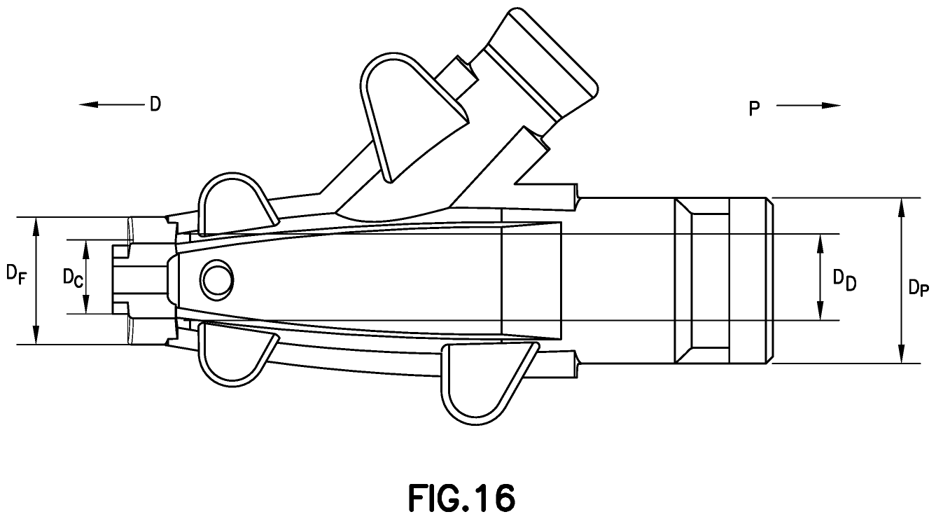

The body 20 of the housing 16 and the access port 22 may be substantially cylindrical. As shown in FIG. 16, the body 20 of the housing 16 may also include a taper from the proximal end 26 to the distal end 28, such that the diameter D_{D} of the distal end 28 of the body 20 of the housing 16 is less than the diameter D_{P} of the proximal end 26 of the body 20 of the housing 16.

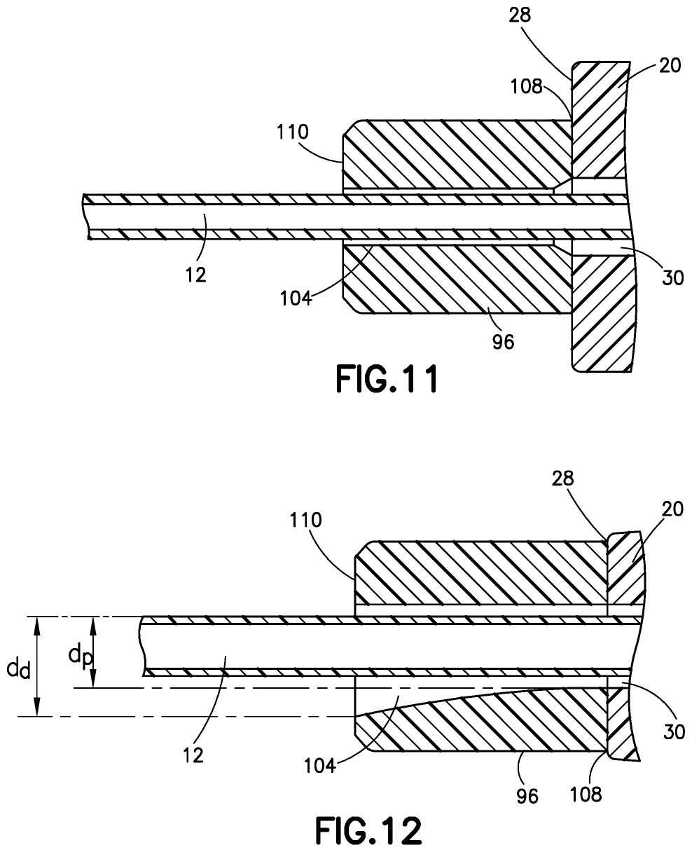

The nose extension portion 96 extends distally beyond the distal end 28 of the housing 16 and defines a passageway 104 (FIG. 11). The catheter 12 passes through the passageway 104 of the nose extension portion 96 and out of the distal end 106 of the catheter adapter 10. The catheter 12 is received within the passageway 104 of the nose extension portion 96 of the catheter adapter 10. The diameter of the passageway 104 of the nose extension portion 96 may be less than the diameter of the opening 30 of the housing 16. An outer surface of the catheter 12 engages at least a portion of the nose extension portion 96 within the passageway 104 of the extension portion 96. Thus, a snug or tight interference between the extension portion 96 and the catheter 12 may be provided.

Referring to FIG. 12, optionally, the passageway 104 of the nose extension portion 96 is tapered from a first diameter dₚ at the proximal end 110 of the nose extension portion 96 to a second diameter d_{d} at the distal end 108 of the nose extension portion 96, with the first diameter dₚ being greater than the second diameter d_{d}. The tapered opening of FIG. 12 is configured to provide greater flexibility of the nose extension portion 96 and the catheter 12.

The body contact portion 98 has an upper surface 114 that corresponds to the outer surface 56 of the housing 16, a lower surface 116 that defines the bottom surface of the catheter adapter 10, which is in contact with the body of the patient when the catheter adapter 10 is in use, and wings 102 extending from opposite sides. The wings 102 may be utilized by a healthcare professional during insertion of the catheter 12 and/or may be used to secure the catheter adaptor 10 to the skin of a patient after insertion of the catheter 12.

The housing 16 is made from a first material and the nose extension portion 96 is made from a second material. The second material is more flexible than the first material. Because the second material of the nose extension portion 96 is more flexible than the first material of the housing 16, the nose extension portion 96 flexes downward toward the skin surface of the patient when the catheter 12 is inserted into the blood vessel 14 of the patient thereby relieving strain on the catheter 12 and preventing sharp bending of the catheter 12. The body contact portion 98 may also be made from the second material.

The first material may be a polymer and the second material may be an elastomer. The second material may have a hardness of 50-70 shore A. The first material may be copolyester, acrylic, polycarbonate, or polypropylene. The second material may be silicone or a thermoplastic elastomer (TPE).

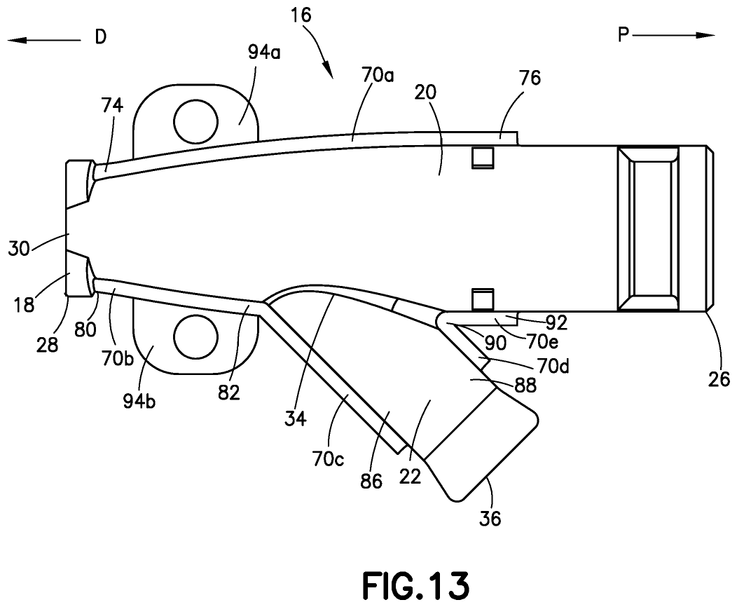

As shown in FIGS. 13-21, an optional extension tip 24 extends from the distal end 28 of the body 20 of the housing 16. The extension tip 24 includes a central portion 40 defining a lumen 42 through which the catheter 12 passes out of the housing 16. The lumen 42 is sized to stably contain the catheter 12 and may have a diameter that is the same as or slightly greater than a diameter of the catheter 12. When the optional extension tip 24 is provided, the nose extension portion 96 surrounds at least a portion of the extension tip 24 and extends distally beyond the distal end 68 of the extension tip 24. The passageway 104 in the nose extension portion 96 is in communication with the lumen 42 in the extension tip 24 of the housing 16.

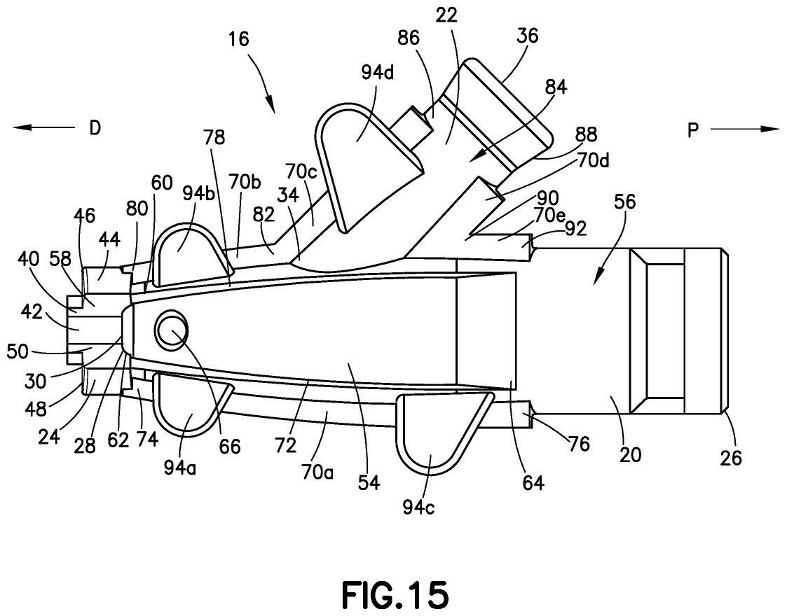

As shown in FIGS. 15-17, a recess 54 may be provided in the outer surface 56 of the bottom side of the body 20 of the housing 16. The recess 54 may extend around a portion of the outer circumference of the body 20 of the housing 16 and may extend along the length of the body 20 of the housing 16 from the distal end 28 of the housing 16 to a location proximal to the access port 22.

At least one protrusion 66 extending from the outer surface 56 of the body 20 of the housing 16 may be provided in the recess 54. As shown in FIGS. 15-17, the protrusion 66 may be provided at a distal end 62 of the recess 54 and may have a substantially circular cross-section. While a single protrusion 66 at the distal end 62 of the recess 54 is described and shown, any number of protrusions 66 may be provided in any position within the recess 54.

As shown in FIGS. 3-10, 13-17, and 20, a flange 70 may be provided that extends laterally from the outer surface 56 of the body 20 of the housing 16. The flange 70 may comprise a plurality of segments 70a-70e. The first flange segment 70a extends longitudinally along the outer surface 56 of the body 20 of the housing 16 on a first longitudinal side 72 of the recess 54 in the outer surface 56 of the body 20 of the housing 16, is circumferentially spaced from first longitudinal side 72 of the recess 54 in the body 20 of the housing 16, has a distal end 74 substantially coincident with the distal end 28 of the body 20 of the housing 16, and has a proximal end 76 substantially coincident with the proximal end 64 of the recess 54 in the body 20 of the housing 16. The second flange segment 70b extends longitudinally along the outer surface 56 of the body 20 of the housing 16 on an opposite second longitudinal side 78 of the recess 54 in the outer surface 56 of the body 20 of the housing 16 from the first flange segment 70a, is circumferentially spaced from the second longitudinal side 78 of the recess 54 in the body 20 of the housing 16, has a distal end 80 substantially coincident with the distal end 28 of the body 20 of the housing 16, and has a proximal end 82 connected to the third flange segment 70c. The third flange segment 70c extends longitudinally along the outer surface 84 of the access port 22 of the housing 16 on a first side 86 of the access port 22. The fourth flange segment 70d extends longitudinally along the outer surface 84 of the access port 22 of the housing 16 on an opposite second side 88 of the access port 22 from the third flange segment 70c. The fifth flange segment 70e extends longitudinally along the outer surface 56 of the body 20 of the housing 16 on the second longitudinal side 78 of the recess 54 in the outer surface 56 of the body 20 of the housing 16, is circumferentially spaced from the second longitudinal side 78 of the recess 54 in the body 20 of the housing 16, has a distal end 90 connected to the fourth flange segment 70d, and has a proximal end 92 substantially coincident with the proximal end 64 of the recess 54 in the outer surface 56 of the body 20 of the housing 16.

As shown in FIGS. 13-17 and 19, a plurality of protrusions 94 extend laterally from the housing 16. The protrusions 94 may extend from the body 20 of the housing 16 and/or the access port 22 of the housing 16. The protrusions 94 extending from the body 20 of the housing 16 are positioned below the flange 70 of the housing 16. If a recess 54 in the outer surface 56 of the body 20 of the housing 16 is provided, the protrusions 94 are located between the flange 70 of the housing 16 and the recess 54 and are circumferentially spaced from the recess 54.

In one embodiment, a first protrusion 94a extends from the body 20 of the housing 16 in an area between the distal end 74 of the first flange segment 70a of the housing 16 and the distal end 62 of the recess 54 in the outer surface 56 of the body 20 of the housing 16. A second protrusion 94b extends from the body 20 of the housing 16 in an area between the distal end 80 of the second flange segment 70b of the housing 16 and the distal end 62 of the recess 54 on the outer surface 56 of the body 20 of the housing 16. The first protrusion 94a and the second protrusion 94b are on opposite sides of the body 20 of the housing 16 and are adjacent the nose extension portion 96. Optionally, third and fourth protrusions 94c, 94d may be provided. The third protrusion 94c extends from the body 20 of the housing 16 in an area adjacent the proximal end 76 of the first flange segment 70a. The fourth protrusion 94d extends laterally from the access port 22 of the housing 16 from a position below the third flange segment 70c of the housing 16.

While the catheter adapter 10 is described and shown as having two or three protrusions 94a-94c extending from the body 20 of the housing 16 and one protrusion 94d extending from the access port 22, any number of protrusions 94 may be provided on the body 20 of the housing 16 and/or the access port 22 of the housing 16. The protrusions 94 may be positioned in any area below the flange 70 of the housing 16. If a recess 54 is provided in the outer surface 56 of the body 20 of the housing 16, the protrusions 94 may be positioned in any area between the flange 70 of the housing 16 and the recess 54.

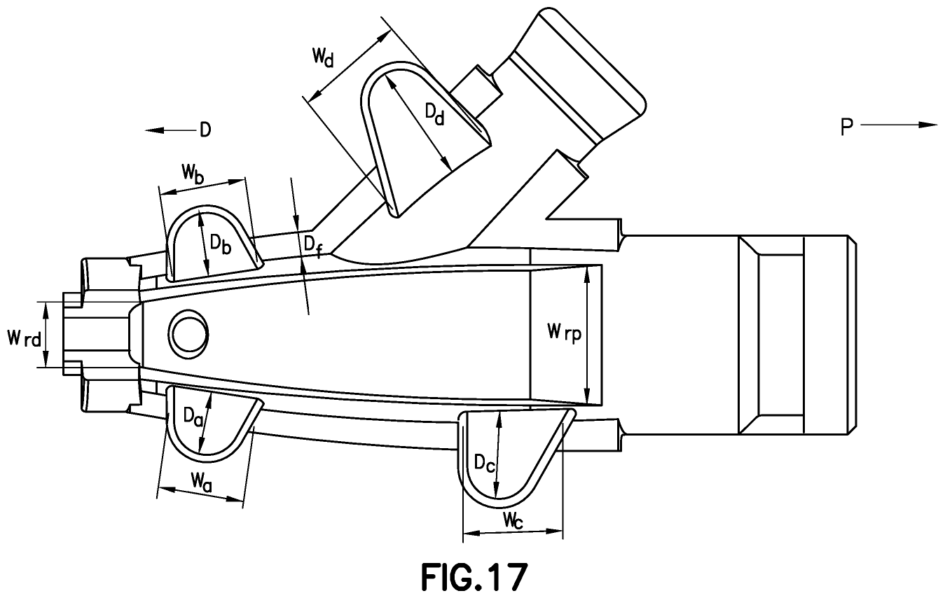

As shown in FIG. 17, the protrusions 94 may have a generally flat, wing-shaped configuration. The lateral distance Dₐ, D_{b}, D_{c}, D_{d} that the wings 94 extend from the housing 16 may be greater than the lateral distance D_{f} that the flange 70 extends from the housing 16. The lateral distance Dₐ, D_{b} that the wings 94a, 94b positioned near the distal end 28 of the body 20 of the housing 16 extend from the housing 16 may be less than the lateral distance D_{c} that the wing 94c positioned nearer the proximal end 26 of the body 20 of the housing 16 and/or the lateral distance D_{d} that the wing 94c positioned on the access port 22 of housing 16 extends from the housing 16. The width Wₐ, W_{b} in the longitudinal direction of the housing 16, of the wings 94a, 94b positioned near the distal end 62 of the body 20 of the housing 16 may be less than the width W_{c}, in the longitudinal direction of the housing 16, of the wing 94c positioned nearer the proximal end 26 of the body 20 of the housing 16 and/or the width W_{d}, in the longitudinal direction of the housing 16, of the wing 94d positioned on the access port 22. The wings 94 may be solid as shown in FIGS. 13-17 and 19 or may include through-holes as shown in FIG. 13.

As can be seen in FIG. 15, all of the flange segments 70 of the housing 16 may lie in a common plane extending parallel to the central longitudinal axis of the housing 16, and all of the protrusions 94 of the housing 16 may lie in a common plane extending parallel to the central longitudinal axis of the housing 16. The plane containing the flange segments 70 of the housing 16 is located above the plane containing the protrusions 94 of the housing 16.

Optionally, as shown in FIGS. 15-19, a flange 44 may surround the central portion 40 of the extension tip 24. As shown in FIG. 16, the central portion 40 of the extension tip 24 may extend distally beyond the flange 44 and may have an outer diameter D_{C} that is less than an outer diameter D_{F} of the flange 44 such that the distal end 46 of the flange 44 defines a ledge 48. The flange 44 may have a diameter D_{F} that is larger than a diameter D_{D} of the distal end 28 of the body 20 of the housing 16.

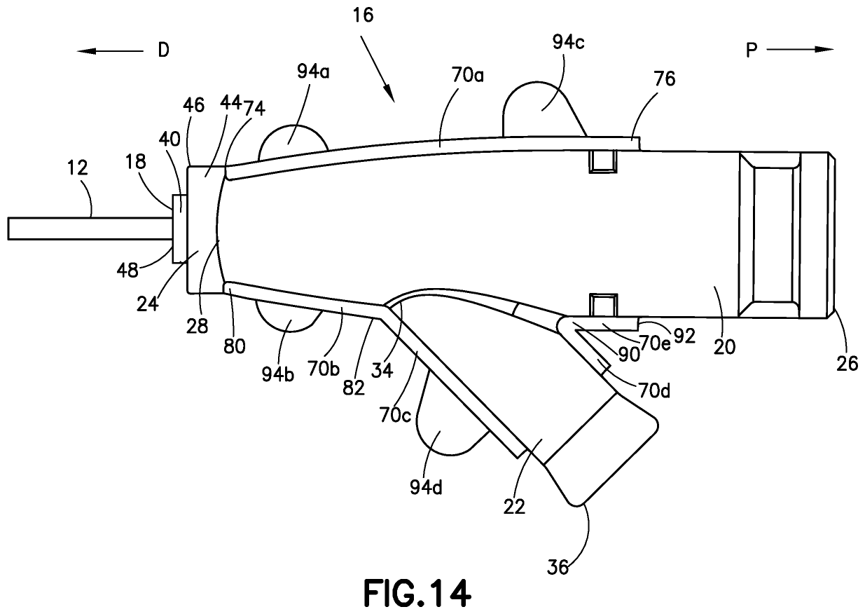

While the nose extension portion 96 and the body contact portion 98 are shown in FIGS. 3-10 as two separate pieces, the nose extension portion 96 and the body contact portion 98 may be linked by a connection portion 100 (FIGS. 14-21)

A notch 50 is provided in a bottom circumference of the distal end 28 of the body 20 of the housing 16 and/or in the optional extension tip 24. The notch 50 may be substantially wedge shaped and may extend through the optional flange 44 and the sidewall of the central portion 40 of the extension tip 24 creating an opening 52 in the sidewall of the central portion 40 of the extension tip 24 that extends into the lumen 42 of the central portion 40 of the extension tip 24. Alternatively, the notch 50 may only extend through the flange 44.

If a recess 54 in the outer surface 56 of the bottom side of the body 20 of the housing 16 is provided, the distal end 62 of the recess 54 may define the notch 50 or the recess 54 may be in communication with the notch 50 in the optional extension tip 24. The recess 54 may extend around the outer circumference of the body 20 of the housing 16 for an angular distance equal to the angular distance defined by the notch 50 in the extension tip 24, such that the sidewall 58 of the notch 50 is continuous with the sidewall 60 of the recess 54, and the circumferential width W_{rd} of the distal end 62 of the recess 54 is less than the circumferential width Wᵣₚ of the proximal end 64 of the recess 54 (FIG. 17). The recess 54 may extend along the length of the body 20 of the housing 16 from the distal end 28 of the body 20 of the housing 16 to a location proximal to the access port 22.

The connection portion 100 connects the nose extension portion 96 to the body contact portion 98. The connection portion 100 extends through and substantially fills the notch 50 in the housing 16. In this manner, the nose extension portion 96 and the body contact portion 98 can be formed as a single, unitary piece in a single forming step.

The body contact portion 98 has an upper surface 114 that corresponds to and fills the area defined beneath the flange 70 of the housing 16 and the outer surface 56 of the housing 16 including the optional recess 54 in the outer surface 56 of the body 20 of the housing 16. The body contact portion 98 further surrounds the protrusions 94 of the housing 16, such that the protrusions 94 of the housing 16 extend into the body contact portion 98 and are completely embedded in the material of the body contact portion 98. The lower surface 116 of the body contact portion 98 defines the bottom surface of the catheter adapter 10 which is in contact with the body of the patient when the catheter adapter 10 is in use.

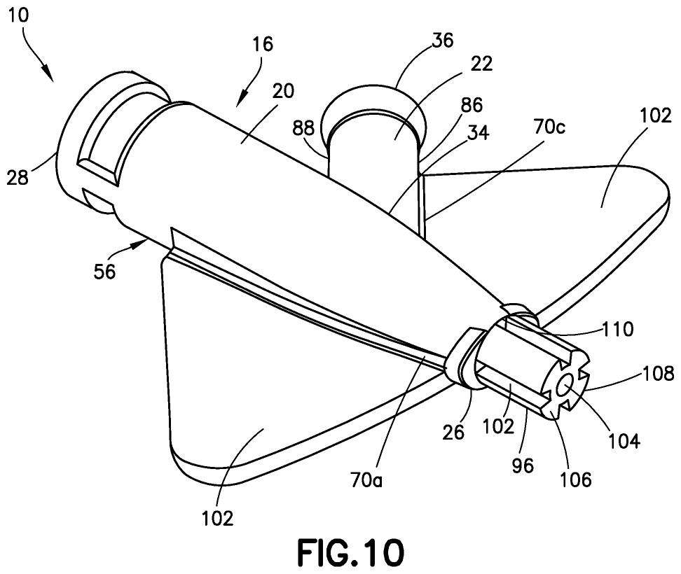

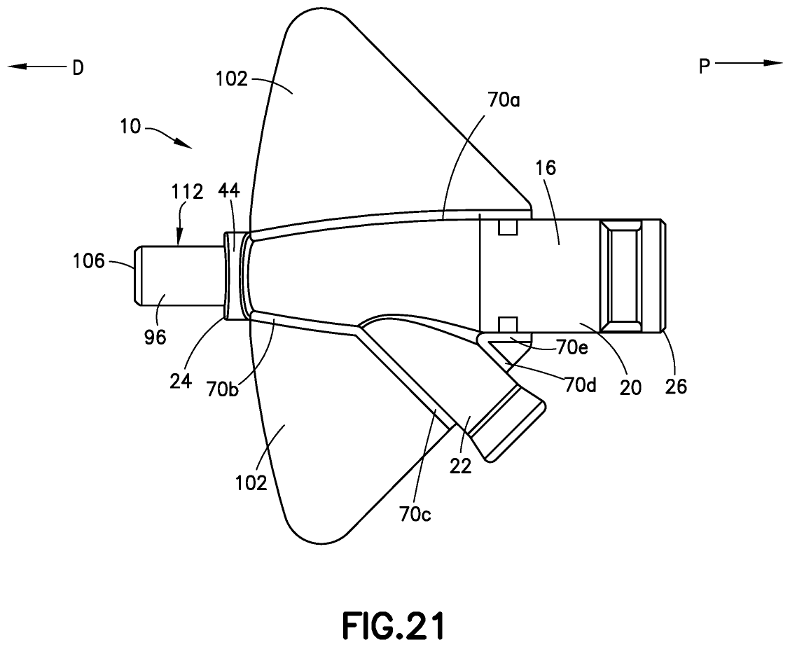

As shown in FIGS. 10 and 21, the material of the body contact portion 98 does not cover the flange 70 of the housing 16. However, in an alternative embodiment, the material of the body contact portion 98 may rise above and cover the flange 70 of the housing 16.

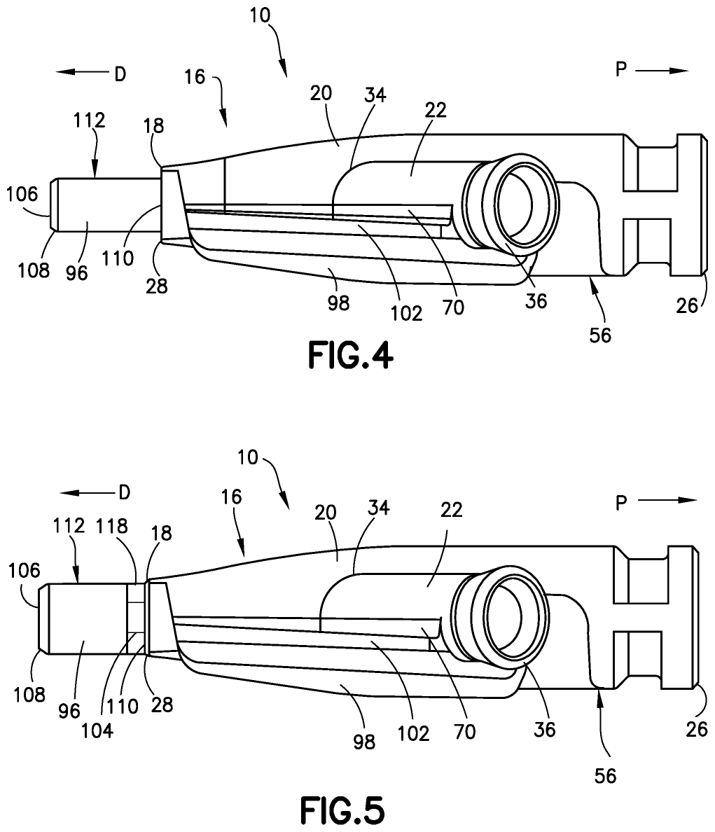

The nose extension portion 96 of the catheter adapter 10 may be substantially cylindrical. The outer diameter of the nose extension portion 96 may be the same as the outer diameter of the distal end 18 of the housing 16 (FIG. 3) or the outer diameter of the nose extension portion 96 may be less than the outer diameter of the distal end 18 of the housing 16 (FIG. 4). For example, the outer diameter of the nose extension portion 96 may be 90-110% of an outer diameter of the distal end 18 of the housing 16.

The nose extension portion 96 and the distal end 18 of the housing 16 form a 90 degree angle, i.e., the outer surface 112 of the nose extension portion 96 is perpendicular to the distal end 18 of the housing 16. The diameter of the nose extension portion 96 may also taper from the proximal end 110 to the distal end 108, such that the diameter of the proximal end 110 of the nose extension portion 96 is greater than the diameter of the distal end 108 of the nose extension portion 96.

As shown in FIG. 5, at least a portion 118 of the nose extension portion 96 may be transparent. The transparent part 118 of the nose extension portion 96 may be formed by the first material of the housing 16 that extends into the nose extension portion 96 or may be an optional flange 44 of the optional extension tip 24. The transparent part 118 and/or the entire housing 16 may be transparent. The transparent part 118 is configured to provide visibility of the catheter 12 to aid with insertion and troubleshooting of the catheter 12 or an instrument inserted within the catheter 12.

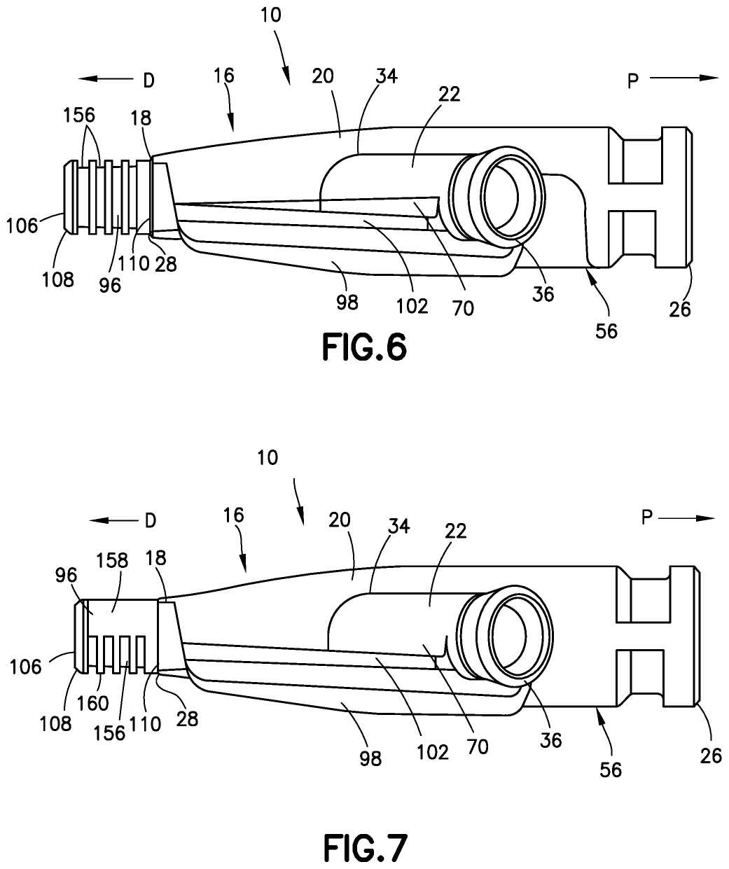

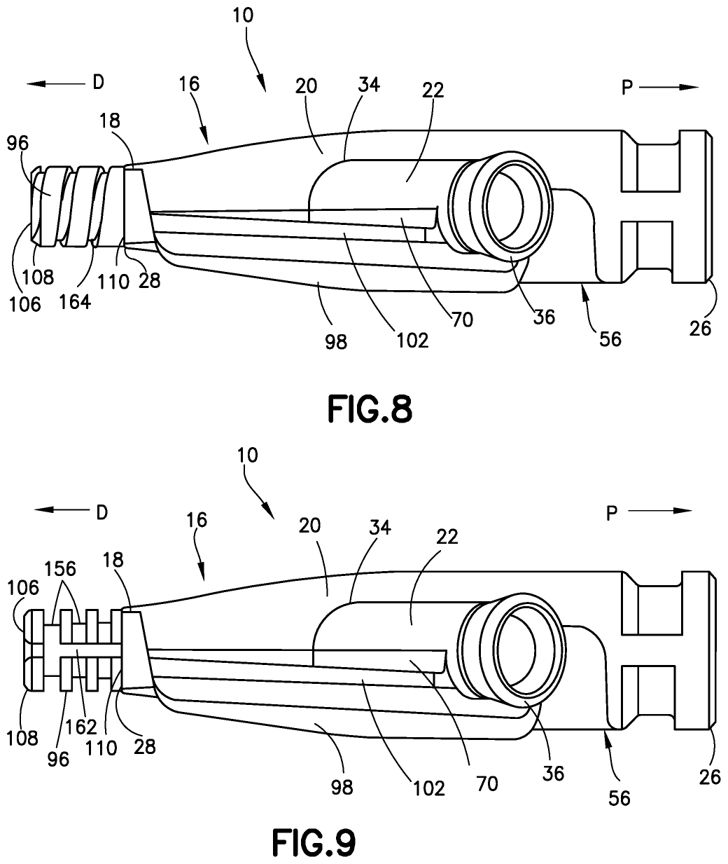

Optionally, (FIGS. 6-10) various features may be provided to increase the flexibility of the nose extension portion 96 while maintaining an outer diameter of the nose extension portion 96 that is the same as or close to an outer diameter of the distal end 18 of the housing 16 of the catheter adapter 10. The increased flexibility may be provided by reducing the cross-sectional area of a portion of the nose extension portion 96 by providing at least one channel in the outer surface 112 of the nose extension portion 96 (FIGS. 6-10). For example, a plurality of radial channels 156 may be provided around all or a portion of the circumference of the outer surface 112 of the nose portion 96 (FIGS. 6 and 7). As shown in FIG. 7, the nose extension portion 96 may include a top portion 158 and a bottom portion 160 positioned opposite the top portion 158, where the plurality of radial channels 156 are only provided on the bottom portion 160 of the nose extension portion 96 to preferentially increase flexibility in the downward direction. Alternatively, axial channels 162, alone (FIG. 10) or in combination with the radial channels 156 (FIG. 9), or a helical channel 164 (FIG. 8), may be provided in the outer surface 112 of the nose portion 96.

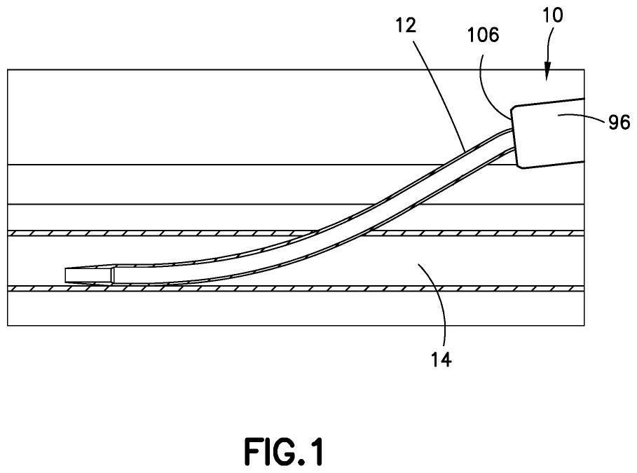

The inventive catheter adapter 10 is configured to provide an optimized angle to minimize kinking, particularly at the S-shaped profile shown in FIG. 1, improve movement of an instrument, such as a line draw device, through the catheter 12, and improve patient comfort by providing a softer, more flexible material for the nose extension portion 96 and the body contact portion 98, which may come into contact with the patient during use of the catheter adapter 10.

The catheter adapter 10 may be injection molded in a two shot process in which the housing 16 is injection molded by injecting the first material into a first mold having a top mold section and a bottom mold section, the bottom mold section corresponding to the shape of the bottom of the housing 16. The first material is solidified in the mold. The mold is opened and the bottom mold section is replaced by a second bottom mold section corresponding to the shape of the nose extension portion 96, the body contact portion 98, and the optional connection portion 100. The second material is injected into the mold and solidified, thereby forming the nose extension portion 96, the body contact portion 98, and the optional connection portion 100 and connecting the nose extension portion 96 and the body contact portion 98 to the housing 16.

The protrusions 94 of the housing 16, the flange 70 of the housing 16, the optional recess 54 in the outer surface 56 of the housing 16, and the optional protrusion 66 in the recess 54, all act to increase the adhesion of the first material of the housing 16 to the second material of the nose extension portion 96 and the body contact portion 98 and to securely connect the housing 16 to the nose extension portion 96 and the body contact portion 98. The connection portion 100 in combination with the notch 50 in the extension tip 24 of the housing 16 allows the nose extension portion 96 and the body contact portion 98 to be formed as a single unitary piece using a one-piece bottom mold in a single forming operation.

Alternatively, the catheter adapter 10 may be injection molded in an over-molding process in which the housing 16 is injection molded by injecting the first material into a first mold. The first material is solidified, and the housing 16 is removed from the mold. The housing 16 is transferred to a second mold corresponding to the shape of t the nose extension portion 96, the body contact portion 98, and the optional connection portion 100. The second material is injected into the mold and solidified, thereby forming the nose extension portion 96 and the body contact portion 98 and connecting the nose extension portion 96 and the body contact portion 98 to the housing 16.

In a further alternative, the nose extension portion 96 and/or the body contact portion 98 may be molded separately from the housing 16 and then attached to the housing 16.

As shown in FIG. 2, the vascular access assembly 120 may include, in addition to the catheter adapter 10 and the catheter 12, a needle assembly 128.

The needle assembly 128 is connected to the proximal end 26 of the housing 16 of the catheter adapter 10 and includes a needle 124 connected to a needle hub 130. The needle 124 passes through the lumen of the catheter 12 and is transitionable from an insertion position in which the distal point 132 of the needle 124 extends beyond the distal end 134 of the catheter 12 to a removal position in which the needle 124 is removed from the catheter 12. A septum (not shown) or other sealing device may be provided in the proximal end 26 of the body 20 of the housing 16 to seal around the needle 124 when the needle 124 is inserted into the body 20 of the housing 16 and/or to seal the passageway 32 of the body 20 of the housing 16 when the needle 124 is completely removed from the housing 16. The needle assembly 128 may include finger grips positioned at the sides of the needle assembly 128 to facilitate various insertion techniques. The needle assembly 128 may further include a needle shield 136 configured to secure the distal point 132 of the needle 124 within the needle shield 136 when the needle assembly 128 is removed from the catheter adapter 10. The needle shield 136 may be activated passively. The needle assembly 128 may further include a push tab 138. The push tab 138 provides a surface for exerting a distal force on the needle assembly 128 to facilitate advancement of the needle 124/catheter 12 after the distal ends of the needle 124/catheter 12 have been inserted into the blood vessel 14 and allows for both one-handed and two-handed advancement. In use, the needle 124, in the insertion position, is used to pierce the skin of the patient and the needle 124 surrounded by the catheter 12 is inserted into the blood vessel 14. Further advancement of the needle 124/catheter 12 is achieved by exerting a distal force on the push tab 138 to urge the needle assembly 128 closer to the patient's skin and the needle 124/catheter 12 further into the blood vessel 14. The needle 124 is then removed from the catheter 12 by pulling the needle hub 130 away from the needle shield 136 in a proximal direction. When the distal point 132 of the needle 124 is received and contained in the needle shield 136, the needle assembly 128 is removed from the catheter adapter 10, the proximal end 26 of the catheter adapter 10 is sealed by the septum, and the catheter adapter 10 is ready for attachment to an intravenous fluid source or a blood collection device.

The vascular access assembly 120 may further include an extension tube 126 connected to the access port 22 and a fluid connector 140. The fluid connector 140 may include a first fluid path 142 having a first connector 144 and a second fluid path 146 having a second connector 148, where both fluid paths 142, 146 are connected to and in fluid communication with the extension tube 126. The first connector 144 may be a luer connector or another needle-free connector, and the first fluid path 142 may include a blood control split septum 150. The second connector 148 may be a luer connector or another needle-free connector, and the second fluid path 146 may include an air vent plug 152. The split septum 150 allows for a reduction in catheter-related bloodstream infection (CRBSI) while providing unrestricted flow and a straight fluid path for the intravenous fluid and functions as a blood control septum. The air vent plug 152 allows air to escape from the system during insertion of the catheter 12, providing continuous confirmation of vascular access while preventing leakage of blood from the system during insertion. After insertion of the catheter 12 into the blood vessel 14, the vent plug 152 is removed from the second fluid path 146 to allow the second fluid path 146 to be connected to an intravenous fluid source via the second connector 148. Alternatively, the fluid connector may have a single fluid path provided with a luer connector or another needle-free connector and an air vent plug that is removed after insertion of the catheter. A clamp 154 may be positioned on the extension tube 126 to prevent blood flow when necessary.

Whereas particular aspects of this invention have been described above for purposes of illustration, it will be evident to those skilled in the art that numerous variations of the details of the present invention may be made without departing from the invention.

The invention claimed is:

1. A catheter adapter comprising:
a housing having a proximal end and a distal end; and
an extension portion extending from the distal end of the housing, the extension portion having a proximal end and a distal end and defining a passageway configured to receive a catheter,
wherein the housing comprises a first material and the extension portion comprises a second material, and the second material is more flexible than the first material.

2. The catheter adapter of claim 1, wherein the first material is a polymer and the second material is an elastomer.

3. The catheter adapter of claim 1, wherein the second material has a hardness of 50-70 shore A.

4. The catheter adapter of claim 1, further comprising a first wing member extending from a first side of the housing and a second wing member extending from a second side of the housing, wherein the first and second wing members are each formed from the second material.

5. The catheter adapter of claim 1, wherein an outer diameter of the extension portion is the same as an outer diameter of the distal end of the housing.

6. The catheter adapter of claim 1, wherein an outer diameter of the extension portion is less than an outer diameter of the distal end of the housing.

7. The catheter adapter of claim 1, wherein the extension portion comprises a plurality of radial channels.

8. The catheter adapter of claim 1, wherein the extension portion comprises a plurality of axial channels.

9. The catheter adapter of claim 7, wherein the extension portion comprises a top portion and a bottom portion positioned opposite the top portion, the bottom portion configured to face a skin surface of a patient, and wherein the plurality of radial channels are only provided on the bottom portion of the extension portion.

10. The catheter adapter of claim 1, wherein the extension portion comprises a helical channel.

11. The catheter adapter of claim 1, further comprising a body contact portion comprising an upper surface corresponding to a surface of the housing and a lower surface defining an outer surface of the catheter adapter.

12. The catheter adapter of claim 1, wherein
the housing comprises:
a body having a proximal end and a distal end;
an access port extending from the body; and
a plurality of protrusions extending from the body and/or the access port;
the body contact portion covers at least a portion of the housing, and
the protrusions are embedded in and are completely surrounded by the body contact portion.

13. The catheter adapter of claim 12, wherein an extension tip extends from the distal end of the body of the housing, and the extension portion surrounds at least a portion of the extension tip.

14. The catheter adapter of claim 13, wherein the extension tip comprises a central portion defining a lumen and a flange that surrounds the central portion, the central portion extending distally beyond the flange.

15. The catheter adapter of claim 13, wherein a notch is provided in the extension tip.

16. The catheter adapter of claim 13, further comprising a connection portion connecting the extension portion to the body contact portion, wherein the connection portion extends through and substantially fills the notch in the extension tip of the housing.

17. The catheter adapter of claim 13, further comprising a recess in an outer surface of the body of the housing in an area covered by the body contact portion.

18. The catheter adapter of claim 17, wherein an extension tip extends from the distal end of the body of the housing, a notch is provided in the extension tip, and the recess is connected to the notch in the extension tip, such that a sidewall of the notch is continuous with a sidewall of the recess.

19. The catheter adapter of claim 18, wherein the recess extends around an outer circumference of the body of the housing for an angular distance equal to an angular distance defined by the notch in the extension tip, and a circumferential width of a distal end of the recess is less than a circumferential width of the proximal end of the recess.

20. The catheter adapter of claim 17, wherein the recess extends along a length of the body of the housing from the notch in the extension portion to a location proximal to the access port.

21. The catheter adapter of claim 17, wherein at least one protrusion extending from the outer surface of the body of the housing is provided in the recess.

22. The catheter adapter of claim 13, further comprising a flange extending laterally from an outer surface of the body of the housing, wherein the body contact portion abuts the flange.

23. The catheter adapter of claim 22, further comprising a recess in an outer surface of the body of the housing in an area covered by the body contact portion, wherein the protrusions extending from the body of the housing are positioned between the flange of the housing and the recess and are circumferentially spaced from the recess.

24. The catheter adapter of claim 12, wherein at least one of the plurality of protrusions extends from the body of the housing and at least one of the plurality of protrusions extends from the access port.

25. The catheter adapter of claim 12, wherein the protrusions have a flat, wing-shaped configuration.

26. The catheter adapter of claim 12, further comprising a flange extending laterally from an outer surface of the body of the housing, wherein the body contact portion abuts the flange, and a lateral distance that the protrusions extend from the housing is greater than a lateral distance that the flange extends from the housing.

27. The catheter adapter of claim 12, further comprising wings extending from opposite sides of the body contact portion.
